(19) United States
(12) Patent Application Publication
(10) Pub. No.: US 2017/0247703 A1
(43) Pub. Date: Aug. 31, 2017

(54) ANTIVIRAL NUCLEASE METHODS

(71) Applicant: Agenovir Corporation, South San Francisco, CA (US)

(72) Inventors: Derek D. Sloan, Belmont, CA (US); Xin Cindy Xiong, San Mateo, CA (US); Stephen R. Quake, Stanford, CA (US)

(21) Appl. No.: 15/442,007

(22) Filed: Feb. 24, 2017

Related U.S. Application Data
(60) Provisional application No. 62/299,839, filed on Feb. 25, 2016.

(51) Int. Cl. C12N 15/113; C12N 15/86; C12N 15/90; C12N 9/22

(52) U.S. Cl. CPC C12N 15/1131; C12N 9/22; C12N 15/1133; C12N 15/1132; C12N 15/86; C12N 15/907

(57) ABSTRACT

Methods and compositions treat a viral infection use a nuclease and an inhibitor that prevents DNA repair, such as a CRISPR-associated nuclease and a small molecule that inhibits an enzyme of a repair pathway. Under guidance of a targeting sequence, the nuclease cuts viral nucleic acid without cutting the patient's genome. The cut ends of the viral nucleic acid are not repaired because the inhibitor prevents a repair mechanism.

### CROSS-REFERENCE TO RELATED APPLICATION

This application claims benefit of U.S. Provisional Application Ser. No. 62/299,839, filed Feb. 25, 2016, incorporated by reference.

### TECHNICAL FIELD

The invention generally relates to compositions and methods for selectively treating viral infections using a guided nuclease system.

### BACKGROUND

Chronic viruses are responsible for infections that can lead to life threatening complications, such as immune systems alterations and even cancer. These persistent viruses often remain in a human host indefinitely, and the infection may transition between symptomatic periods and latent periods. Many chronic viral infections are linked to cancer. For example, high-risk HPV is able to integrate into the host DNA and is thought to cause cancer by inactivating tumor suppressors within the host DNA. The Epstein-Barr virus (EBV) is directly associated with cancers (such as Hodgkin's and Burkitt's lymphomas) due to its presence at various stages of B-cell development.

The link between oncoviruses and cancers has led to the development of vaccines and other therapies to eliminate the infection and accompanying cancer. Vaccines, however, are only successful against viruses if they are administered before the person is infected. There have been a few other potential antiviral therapies directed at oncoviruses but none have been successful.

### SUMMARY

The invention provides compositions and methods for treating a viral infection in a patient by selectively cleaving viral nucleic acid and preventing subsequent repair of the viral nucleic acid. A nuclease, such as Cas9, and a DNA repair inhibitor are delivered to infected cells. The nuclease specifically cuts the viral nucleic acid (e.g., under the guidance of a guide RNA that does not have any match in a human genome). The inhibitor prevents a repair mechanism such as end-joining, synthesis, or ligation. The combination of gene editing and inhibition of viral nucleic acid repair act in concert to reduce or eliminate the effects of the viral infection. In the case of an oncovirus, this means the reduction or elimination of the oncogenic effects of the virus. The combination preferably works without disrupting host genomic material (i.e., other than integrated viral sequence). The invention works on integrated as well as non-integrated virus and is equally effective on latent and active virus.

Combination therapies of the invention preferably include a nuclease, such as Cas9 or a Cas9 variant that is targeted toward oncoviral sequence. It is recognized however, that any targeted endonuclease is useful including, but not limited to, Cas6, Cas5, Cfp1, a zinc finger nuclease (ZFN), a meganuclease, a transcription activator-like effector nuclease (TALEN), or a variant of any of the foregoing. In addition to the nuclease component, there is a component that is useful in inhibiting ligation of viral sequence that has been cleaved. A ligation inhibitor may be a small molecule that prevents end-joining repair, an enzyme that removes a 5′ phosphate or 3′ hydroxyl, an enzyme that adds blocking groups or fragments of DNA that are blocked or that lack an accessible 5′ phosphate or 3′ hydroxyl, or other such moieties. Repair can be inhibited by inhibiting an end-joining repair pathway or by interfering with synthesis or ligation, e.g., by preventing function of a synthetase or a ligase. Additionally or alternatively, repair may be inhibited by enhancing cell exonuclease activity, e.g. to increase degradation of SSB and DSB (single and double strand DNA breaks). For example, human exonuclease 1 (hEXO1) efficiently repairs DSB. hEXO1 is ubiquitinated and degraded in the proteasome. Thus, in some embodiments, a combination of a targeted endonuclease with a proteasome inhibitor are delivered to enhance hEXO-1 activity and synergize to kill viral DNA+cells.

The inhibitor may be co-delivered with the targeted nuclease to suppress activation of homologous or non-homologous end repair mechanisms in the resulting fragments. End repair mechanisms may be inhibited, for example, by a treatment that includes a small molecule such as KU55933, caffeine, or wortmannin.

In certain aspects, the invention provides a system for treating cells with a viral infection, e.g. cells that contain viral nucleic acid. The system includes a nuclease, a targeting sequence, and a DNA repair inhibitor. The target nucleic acid is typically viral nucleic acid. However, any appropriate nucleic acid may be targeted. In preferred embodiments, the system is used to degrade any foreign nucleic acid including, for example, sequences from intracellular parasites such as malaria or intracellular bacteria or mycobacteria such as tuberculosis. The targeting sequence directs the nuclease to the viral nucleic acid, the nuclease cuts the viral nucleic acid into fragments, and the inhibitor prevents repair of the fragments. The targeting sequence may include one or more guide RNAs. The nuclease may include one or more of a zinc-finger nuclease, a TALENs nuclease, a meganuclease, a Cas9 endonuclease, or others known in the art. Preferably, the nuclease is Cas9, encoded along with a guide RNA that specifically targets the target nucleic acid. In a preferred embodiment, the nuclease is obtained or delivered in a ribonucleoprotein (RNP) form, e.g. as a recombinant Cas9 protein duplexed with sgRNA or with crRNA+tracRNA, or as a recombinant TALEN protein. It may be found that delivery as RNP is more effective and less toxic than plasmid DNA, and that RNP permits delivery of pre-formed enzymatically active drug (which acts faster), and is only active in the cell for a very limited time (<24 hours), thus reducing non-specific toxicity and off-target activity. RNP can be directly electroporated into primary tissues, e.g. peripheral blood mononuclear cells (PBMCs), for ex vivo transplant indications. RNP, like mRNA or pDNA, can also be incorporated into cationic lipid nanoparticles for in vivo delivery indications, e.g. cancer. In certain embodiments, the inhibitor prevents homologous or non-homologous end repair of the one or more fragments. For example, the treatment may be a small molecule such as KU55933, caffeine, VE-821, NU6027, UNC-01, mirin, RI-1, streptonigrin, RI-2, 3-ABA, olaparib, NU1025, NSC130813, wortmannin, NU7026, SCR7, or L189. Additionally or alternatively, the inhibitor may include an enzyme or protein that functions to inhibit components essential to end-repair processes. In one example, the treatment may include the enzyme Antarctic phosphatase, which removes the 5′ phosphate from DNA and RNA ends. In some embodiments, it is recognized that the double-stranded breaks (DSBs) introduced by Cas-type nuclease are primarily repaired via non-homologous end joining (NHEJ) and that DNA ligase IV (LIG4) is critical for NHEJ. Other LIGs (1-3) are involved in repair of SSB and DSB. Systems and methods described herein may include one or more small molecule inhibitors of LIG4 or other LIGs. For example, the compound L82 has been identified as an uncompetitive inhibitor of DNA ligase I. L67 is a compound that inhibits LIG1 and LIG3. Other compounds that have been identified as inhibitors of a DNA Ligase may be used. Additionally or alternatively, the inhibitor may include the delivery of siRNAs that inhibit the function of DNA ligase or other enzymes that involved in repair process.

In other embodiments, the inhibitor includes a nucleic acid fragment that is ligated to an exposed viral end, wherein the newly-added end (provided by the inhibitor) lacks either or a 5′ phosphate or a 3′ hydroxyl (e.g., it may provide a chain-terminating nucleotide). In certain embodiments, the inhibitor includes one or more dideoxy-nucleotides, which terminate nucleic acid synthesis when incorporated. The inhibitor may include an enzyme that removes or that blocks a 3′ hydroxyl or 5′ phosphate. Any enzyme or moiety that results in fragments lacking fragment ends that are accessible for ligation or polymerization may be used.

Aspects of the invention provide methods for treating cells infected with a virus. Methods includes obtaining a nuclease that is designed to cut a target viral nucleic acid and an inhibitor that prevents repair of the cut viral nucleic acid. Preferably, the nuclease cuts the viral nucleic acid without cutting a portion of the human genome important for normal cellular function. Suitable targets in viral genomes include, but are not limited to, a portion of a genome or gene of a hepatitis virus, a hepatitis B virus (HBV), an Epstein-Barr virus, a Kaposi's sarcoma-associated herpesvirus (KSHV), a herpes-simplex virus (HSV), a cytomegalovirus (CMV), human papilloma virus (HPV), and Merkel cell polyomavirus. The target in the viral genome may lie within one or more of a preC promoter in a hepatitis B virus (HBV) genome, an S1 promoter in the HBV genome, an S2 promoter in the HBV genome, an X promoter in the HBV genome, a viral Cp (C promoter) in an Epstein-Barr virus genome, a minor transcript promoter region in a Kaposi's sarcoma-associated herpesvirus (KSHV) genome, a major transcript promoter in the KSHV genome, an Egr-1 promoter from a herpes-simplex virus (HSV), an ICP 4 promoter from HSV-1, an ICP 10 promoter from HSV-2, a cytomegalovirus (CMV) early enhancer element, a cytomegalovirus immediate-early promoter, an HPV early promoter, and an HPV late promoter.

In a preferred embodiment, the nuclease is obtained or delivered in a ribonucleoprotein (RNP) form, e.g. as a recombinant Cas9 protein duplexed with sgRNA or with crRNA+tracRNA, or as a recombinant TALEN protein. It may be found that delivery as RNP is more effective and less toxic than plasmid DNA, and that RNP permits delivery of pre-formed enzymatically active drug (which acts faster), and is only active in the cell for a very limited time (<24 hours), thus reducing non-specific toxicity and off-target activity. RNP can be directly electroporated into primary tissues, e.g. peripheral blood mononuclear cells (PBMCs), for ex vivo transplant indications. RNP, like mRNA or pDNA, can also be incorporated into cationic lipid nanoparticles for in vivo delivery indications, e.g. cancer.

The invention may further involve one or more vectors or carriers for delivering the nuclease, targeting sequence, inhibitor, or combination thereof into cells of a patient. In certain embodiments, a vector, such as a plasmid, encodes any one or more of the nuclease, the targeting sequence, and the inhibitor. In other embodiments, a first vector encodes the nuclease and the targeting sequence, and a second vector encodes the inhibitor. In certain embodiments, the nuclease and optionally a targeting sequence such as a gRNA or sgRNA are encoded on a vector such as a plasmid, and the treatment is a small molecule. Suitable non-viral vectors include a plasmid, a nanoparticle, a cationic lipid, a cationic polymer, a metallic nanopolymer, a nanorod, a liposome, a micelle, a microbubble, a cell-penetrating peptide, and a liposphere. In some instances, the vector may be a viral vector. Suitable viral vectors include retrovirus, lentivirus, adenovirus, herpes virus, pox virus, alpha virus, and adeno-associated viruses.

In preferred embodiments, a vector that encodes the nuclease also encodes the targeting sequence, which then guides the nuclease to a target on a genome of a virus. The targeting sequence is typically a guide RNA. The targeting sequence preferably matches the target according to a predetermined criteria and does not match any portion of a host genome according to the predetermined criteria (e.g., is at least 60% complementary within a 20 nucleotide stretch and presence of a protospacer adjacent motif adjacent the 20 nucleotide stretch). The guide sequence should not match any portion of the host genome (e.g., human genome) according to the predetermined criteria.

Alternatively, compositions of the invention may be delivered via a liposome, a cell-penetrating peptide, a nanoparticle, polymers, glycopolymers, transfection, electroporation, or any other suitable carrier or technique.

In some aspects, the invention provides a pharmaceutical composition comprising any of the nucleic acids described above. The pharmaceutical composition may include a transfection-facilitating cationic lipid formulation. The pharmaceutical composition includes appropriate diluents, adjuvants, and carriers for delivering the active components to targeted cells. The carrier may be, for example, a liposome, a nanoparticle, a peptide, a polymer, a lipid, or a nanoplex. The formulation may include standard pharmacologic formulations, including timed release formulations and other well-known pharmaceutical formulations.

In related aspects, the invention provides for the use of any of the compounds or molecules described above in the manufacture of a medicament for treatment of a viral infection, preferably a latent viral infection.

### BRIEF DESCRIPTION OF DRAWINGS

FIG. 1 diagrams a method of the invention.

FIG. 2 shows a nucleic acid that encodes a nuclease, a targeting sequence and an inhibitor of DNA repair.

FIG. 3 shows a plasmid according to certain embodiments.

FIG. 4 shows the results of successfully cleaving the HPV genome using Cas9 endonuclease, a gRNA for E6, and a gRNA for E7.

FIG. 5 shows a gel resulting from an in vitro CRISPR assay against HBV.

FIG. 6 shows a plasmid according to certain embodiments.

FIG. 7 diagrams the EBV genome.

FIG. 8 shows genomic context around guide RNA sgEBV2 and PCR primer locations.

FIG. 9 shows a large deletion induced by targeting sgEBV2.

FIG. 10 shows that sequencing confirmed the connection of expected cutting sites.

FIG. 11 shows three small molecule inhibitors of DNA ligase.

### DETAILED DESCRIPTION

An infection is treated by delivering a nuclease that cuts viral nucleic acid and an inhibitor that prevents repair of the cut viral nucleic acid. Any suitable nuclease can be used. Where a CRISPR-associated (Cas)-type nuclease (e.g., Cas5, Cas6, Cas9, Cfp1, or a modified version thereof) is used, the Cas-type nuclease is delivered along with an RNA that targets the nuclease to the viral nucleic acid. Any suitable inhibitor may be used such as, for example, a small molecule drug, an enzyme, or other molecular entity. Small molecules that inhibit enzymes of a DNA repair pathway are known and may be used. Additionally or alternatively, the inhibitor may be provided by an enzyme that modifies a free end of the cut nucleic acid so that it is not accessible for a repair. The inhibitor may be a nucleotide or nucleoside analog or ddNTP that prevents a successful repair.

The nuclease may be initially provided for delivery in any suitable form. For example, the nuclease may be delivered as an active enzyme or ribonucleoprotein (RNP) or the nuclease may be encoded in a nucleic acid, such as in a DNA vector or as mRNA. Likewise, where the inhibitor is a protein, the inhibitor may initially be provided in any suitable form such as a protein or encoded in a nucleic acid. Where the nuclease and the inhibitor are to be provided in a nucleic acid form, they may both be encoded on the same nucleic acid (e.g., DNA plasmid or mRNA) with or without a spacer or linker, or they may be separately delivered.

The nuclease and the inhibitor may be delivered to the infected cells together (e.g., as part of a single composition) or they may be delivered separately, wholly or partially simultaneously or separately. Either or both of the nuclease and inhibitor may be provided with a pharmaceutically acceptable carrier or prepared for delivery orally, intravenously, topically, or by any suitable method. Either or both of the nuclease and the inhibitor may be delivered using a suitable viral or non-viral vector or delivery method or other suitable format. For example, the nuclease, targeting sequence, and the treatment may be delivered on the same vehicle, whether nucleic acid, plasmid, or viral vector. Alternatively, the nuclease and targeting sequence may be delivered in one manner, and the treatment may be delivered in a separate manner. For example, a cocktail may include: (i) a vector encoding the nuclease and the targeting sequence; and (ii) the treatment or a vector encoding the treatment. In one embodiment, the delivery method includes the use of ribonucleoproteins (RNP). For example, the ribonucleotide may include Cas9 (as the protein) and guide RNA as the ribonucleic acid. Delivery as RNP allows control over dosing and avoids continuous production of nuclease proteins by the cell. In some embodiments, mRNA may be used to deliver the nuclease, to encourage continued production of the nuclease.

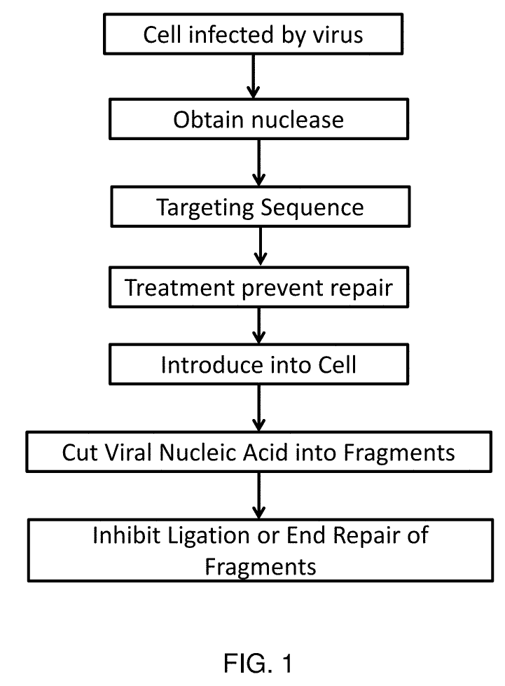

FIG. 1 diagrams a method of treating a viral infection. Methods of the invention are applicable to in vivo treatment of patients and may be used to remove any viral genetic material, such as genes of virus associated with a latent viral infection. Methods may be used in vitro, or ex vivo, e.g., to prepare or treat a cell culture or cell sample. When used in vivo, the cell may be any suitable germ line or somatic cell and compositions of the invention may be delivered to specific parts of a patient's body or be delivered systemically. If delivered systemically, it may be preferable to include within compositions of the invention tissue-specific promoters. For example, if a latent viral infection is localized to the liver, hepatic tissue-specific promotors may be included in a plasmid or viral vector that codes for a targeted nuclease.

Methods of the invention are suitable for the treatment of viruses, including, but not limited to, the following viruses: adenovirus, herpes simplex virus, varicella-zoster virus, Epstein-Barr virus, human cytomegalovirus, human herpesvirus type 8, human papillomavirus, BK virus, JC virus, smallpox, hepatitis B virus, human bocavirus, parvovirus, B19, human astrovirus, Norwalk virus, coxsackievirus, hepatitis A virus, poliovirus, rhinovirus, sever acute respiratory syndrome virus, hepatitis C virus, yellow fever virus, dengue virus, west nile virus, rubella virus, hepatitis E virus, human immunodeficiency virus, influenza virus, guanarito virus, junin virus, lassa virus, machupo virus, sabia virus, Crimean-Congo hemorrhagic fever virus, ebola virus, Marburg virus, measles virus, mumps virus, parainfluenza virus, respiratory syncytial virus, human metapnemovirus, Hendra virus, nipah virus, rabies virus, hepatitis D virus, rotavirus, orbivirus, coltivirus, or banna virus.

Methods of the invention involve obtaining a nuclease that is designed to cut or cleave a target nucleic acid. Typically, the target nucleic acid is viral nucleic acid. Suitable targets in viral genomes include, for example, a portion of a genome or gene of a hepatitis virus, a hepatitis B virus (HBV), an Epstein-Barr virus, a Kaposi's sarcoma-associated herpesvirus (KSHV), a herpes-simplex virus (HSV), a cytomegalovirus (CMV), and a human papilloma virus (HPV). The target in the viral genome may lie within one or more of a preC promoter in a hepatitis B virus (HBV) genome, an S1 promoter in the HBV genome, an S2 promoter in the HBV genome, an X promoter in the HBV genome, a viral Cp (C promoter) in an Epstein-Barr virus genome, a minor transcript promoter region in a Kaposi's sarcoma-associated herpesvirus (KSHV) genome, a major transcript promoter in the KSHV genome, an Egr-1 promoter from a herpes-simplex virus (HSV), an ICP 4 promoter from HSV-1, an ICP 10 promoter from HSV-2, a cytomegalovirus (CMV) early enhancer element, a cytomegalovirus immediate-early promoter, an HPV early promoter, and an HPV late promoter.

While methods of the invention may be used to target and treat viruses, methods of invention may also be used to directly treat mutated or tumor nucleic acid. For example, methods and systems of the invention may target gene signatures unique to tumors. The gene signature unique to a tumor may include a signature related to proliferation of tumor nucleic acid or may include a signature directly related to the responsiveness of the tumor to chemotherapy or other medicinal treatments. For example, tumors with Ras mutations have been found less responsive to chemotherapy than tumors with normal Ras. In such aspects, methods of the invention may target a nuclease to Ras-mutated tumor nucleic acid, use the nuclease to cut the Ras-mutated tumor nucleic acid into fragments, and then use a molecule or moiety to inhibit repair of the ends of the fragments (e.g., by treatment with Antarctic phosphatase, or by ligating fragments with dideoxy ends to 5′ ends of the fragments). Such a treatment destroys the Ras-mutated tumor nucleic acid. With the Ras-mutated tumor nucleic acid destroyed, the tumor may be more receptive to, for example, chemotherapy.

Systems and methods of the invention include one or more nucleases, one or more guide or targeting sequences, and one or more inhibitor of DNA repair. The nuclease is designed to cut or cleave target nucleic acid (such as viral nucleic acid) into fragments, and the guide sequence targets the nuclease to a viral genomic target. The inhibitor prevents ligation of the resulting fragments or nucleic acid synthesis. In an illustrative embodiment, the nuclease and the inhibitor are both provided encoded on a plasmid to be transcribed and translated in the infected cells.

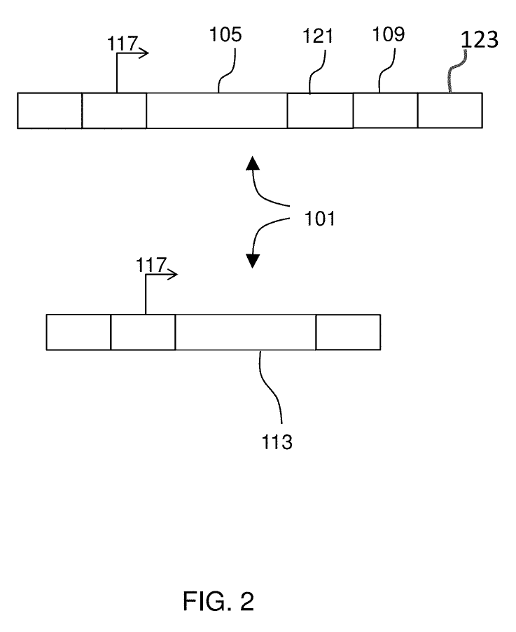

FIG. 2 shows nucleic acid 101 that encodes a nuclease 105, a guide or targeting sequence 121, and an inhibitor 109. In the depicted embodiment, the inhibitor is an enzyme that prevents repair of cut DNA. For example, the inhibitor may be Antarctic phosphatase. Other features may optionally be included in the nucleic acid 101. For example, the nucleic acid may further include a switch 113 that causes the nuclease to be expressed in the presence of a viral nucleic acid (a riboswitch). The nucleic acid 101 may include one or more promoter 117 to aid in transcription of the included genes. Additionally, the nucleic acid 101 may include a portion that codes for a nuclear localization signal 123 so that the nuclease 105, the inhibitor, or both, when expressed by transcription and translation, are tagged for import into the nucleus of a host cell so that they can attack the viral DNA there.

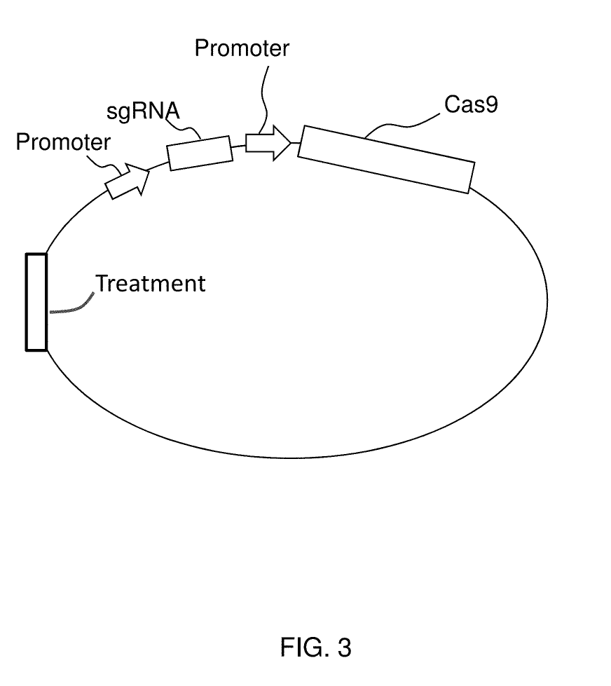

FIG. 3 shows a composition for treating a viral infection according to certain embodiments. The composition preferably includes a vector (which may be a plasmid, or a viral vector) that codes for a nuclease that cuts viral nucleic acid into fragments, a targeting sequence (e.g., a gRNA) that targets the nuclease to viral nucleic acid, and a treatment that prevents DNA repair or ligation of the fragments. The composition may optionally include one or more of a promoter, replication origin, other elements, or combinations thereof as described further herein.

### Nuclease

Systems and methods of the invention include using a programmable or targetable nuclease to specifically target viral nucleic acid for destruction. Any suitable targeting nuclease can be used including, for example, zinc-finger nucleases (ZFNs), transcription activator-like effector nucleases (TALENs), clustered regularly interspaced short palindromic repeat (CRISPR)-associated (Cas) nucleases, meganucleases, other endo- or exo-nucleases, or combinations thereof. See Schiffer, 2012, Targeted DNA mutagenesis for the cure of chronic viral infections, J Virol 88(17):8920-8936, incorporated by reference.

Cas-type nucleases are nucleases that complex with small RNAs as guides (gRNAs) to cleave DNA in a sequence-specific manner upstream of the protospacer adjacent motif (PAM) in a target location. A Cas-type nuclease may use separate guide RNAs known as the crRNA and tracrRNA. These two separate RNAs have been combined into a single RNA to enable site-specific mammalian genome cutting through the design of a short guide RNA. Cas9 and guide RNA (gRNA) may be synthesized by known methods. Cas9/guide-RNA (gRNA) uses a non-specific DNA cleavage protein Cas9, and an RNA oligo to hybridize to target and recruit the Cas9/gRNA complex. See Chang et al., 2013, Genome editing with RNA-guided Cas9 nuclease in zebrafish embryos, Cell Res 23:465-472; Hwang et al., 2013, Efficient genome editing in zebrafish using a CRISPR-Cas system, Nat. Biotechnol 31:227-229; Xiao et al., 2013, Chromosomal deletions and inversions mediated by TALENS and CRISPR/Cas in zebrafish, Nucl Acids Res 1-11.

CRISPR (Clustered Regularly Interspaced Short Palindromic Repeats) is found in bacteria and is believed to protect the bacteria from phage infection. It has recently been used as a means to alter gene expression in eukaryotic DNA, but has not been proposed as an anti-viral therapy or more broadly as a way to disrupt genomic material. Rather, it has been used to introduce insertions or deletions as a way of increasing or decreasing transcription in the DNA of a targeted cell or population of cells. See for example, Horvath et al., Science (2010) 327:167-170; Terns et al., Current Opinion in Microbiology (2011) 14:321-327; Bhaya et al. Annu Rev Genet (2011) 45:273-297; Wiedenheft et al. Nature (2012) 482:331-338); Jinek M et al. Science (2012) 337:816-821; Cong L et al. Science (2013) 339:819-823; Jinek M et al. (2013) eLife 2:e00471; Mali Pet al. (2013) Science 339:823-826; Qi L S et al. (2013) Cell 152:1173-1183; Gilbert L A et al. (2013) Cell 154:442-451; Yang H et al. (2013) Cell 154:1370-1379; and Wang H et al. (2013) Cell 153:910-918).

In an aspect of the invention, the Cas9 endonuclease causes a double strand break in one or more locations in viral nucleic acid and the inhibitor prevents repair. The result is that the host cell will be free of viral infection.

In embodiments of the invention, nucleases cleave the genome of the target virus. A nuclease is an enzyme capable of cleaving the phosphodiester bonds between the nucleotide subunits of nucleic acids. Endonucleases are enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, cleave only at very specific nucleotide sequences. In a preferred embodiment of the invention, the Cas9 nuclease is incorporated into the compositions and methods of the invention, however, it should be appreciated that any nuclease may be utilized.

In preferred embodiments of the invention, the Cas9 nuclease is used to cleave the genome. The Cas9 nuclease is capable of creating a double strand break in the genome. The Cas9 nuclease has two functional domains: RuvC and HNH, each cutting a different strand. When both of these domains are active, the Cas9 causes double strand breaks in the genome.

TALENs uses a nonspecific DNA-cleaving nuclease fused to a DNA-binding domain that can be to target essentially any sequence. For TALEN technology, target sites are identified and expression vectors are made. Linearized expression vectors (e.g., by Notl) may be used as template for mRNA synthesis. A commercially available kit may be use such as the mMESSAGE mMACHINE SP6 transcription kit from Life Technologies (Carlsbad, Calif.). See Joung & Sander, 2013, TALENs: a widely applicable technology for targeted genome editing, Nat Rev Mol Cell Bio 14:49-55.

TALENs and CRISPR methods provide one-to-one relationship to the target sites, i.e. one unit of the tandem repeat in the TALE domain recognizes one nucleotide in the target site, and the crRNA, gRNA, or sgRNA of CRISPR/Cas system hybridizes to the complementary sequence in the DNA target. Methods can include using a pair of TALENs or a Cas9 protein with one gRNA to generate double-strand breaks in the target.

ZFN may be used to cut viral nucleic acid. Briefly, the ZFN method includes introducing into the infected host cell at least one vector (e.g., RNA molecule) encoding a targeted ZFN 305 and, optionally, at least one accessory polynucleotide. See, e.g., U.S. Pub. 2011/0023144 to Weinstein, incorporated by reference. The cell includes target sequence. The cell is incubated to allow expression of the ZFN, wherein a double-stranded break is introduced into the targeted chromosomal sequence by the ZFN. In some embodiments, a donor polynucleotide or exchange polynucleotide is introduced. Swapping a portion of the viral nucleic acid with irrelevant sequence can fully interfere transcription or replication of the viral nucleic acid.

Typically, a ZFN comprises a DNA binding domain (i.e., zinc finger) and a cleavage domain (i.e., nuclease) and this gene may be introduced as mRNA (e.g., 5′ capped, polyadenylated, or both). Zinc finger binding domains may be engineered to recognize and bind to any nucleic acid sequence of choice. See, e.g., Qu et al., 2013, Zinc-finger-nucleases mediate specific and efficient excision of HIV-1 proviral DAN from infected and latently infected human T cells, Nucl Ac Res 41(16):7771-7782, incorporated by reference. An engineered zinc finger binding domain may have a novel binding specificity compared to a naturally-occurring zinc finger protein. Engineering methods include, but are not limited to, rational design and various types of selection. A zinc finger binding domain may be designed to recognize a target DNA sequence via zinc finger recognition regions (i.e., zinc fingers). See for example, U.S. Pat. Nos. 6,607,882; 6,534,261 and 6,453,242, incorporated by reference. Exemplary methods of selecting a zinc finger recognition region may include phage display and two-hybrid systems, and are disclosed in U.S. Pat. No. 5,789,538; U.S. Pat. No. 5,925,523; U.S. Pat. No. 6,007,988; U.S. Pat. No. 6,013,453; U.S. Pat. No. 6,410,248; U.S. Pat. No. 6,140,466; U.S. Pat. No. 6,200,759; and U.S. Pat. No. 6,242,568, each of which is incorporated by reference.

A ZFN also includes a cleavage domain. The cleavage domain portion of the ZFNs may be obtained from any suitable endonuclease or exonuclease such as restriction endonucleases and homing endonucleases. See, for example, Belfort & Roberts, 1997, Homing endonucleases: keeping the house in order, Nucleic Acids Res 25(17):3379-3388. A cleavage domain may be derived from an enzyme that requires dimerization for cleavage activity. Two ZFNs may be required for cleavage, as each nuclease comprises a monomer of the active enzyme dimer. Alternatively, a single ZFN may comprise both monomers to create an active enzyme dimer. Restriction endonucleases present may be capable of sequence-specific binding and cleavage of DNA at or near the site of binding. Certain restriction enzymes (e.g., Type IIS) cleave DNA at sites removed from the recognition site and have separable binding and cleavage domains. For example, the Type IIS enzyme FokI, active as a dimer, catalyzes double-stranded cleavage of DNA, at 9 nucleotides from its recognition site on one strand and 13 nucleotides from its recognition site on the other. The FokI enzyme used in a ZFN may be considered a cleavage monomer. Thus, for targeted double-stranded cleavage using a FokI cleavage domain, two ZFNs, each comprising a FokI cleavage monomer, may be used to reconstitute an active enzyme dimer. See Wah, et al., 1998, Structure of FokI has implications for DNA cleavage, PNAS 95:10564-10569; U.S. Pat. No. 5,356,802; U.S. Pat. No. 5,436,150; U.S. Pat. No. 5,487,994; U.S. Pub. 2005/0064474; U.S. Pub. 2006/0188987; and U.S. Pub. 2008/0131962, each incorporated by reference.

Meganucleases are endodeoxyribonucleases characterized by a large recognition site (double-stranded DNA sequences of 12 to 40 base pairs); as a result this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes. Meganucleases can be divided into five families based on sequence and structure motifs. Crystal structures illustrates mode of sequence specificity and cleavage mechanism for meganucleases: (i) specificity contacts arise from the burial of extended (β-strands into the major groove of the DNA, with the DNA binding saddle having a pitch and contour mimicking the helical twist of the DNA; (ii) full hydrogen bonding potential between the protein and DNA is never fully realized; (iii) cleavage to generate 4-nt 3′-OH overhangs occurs across the minor groove, wherein the scissile phosphate bonds are brought closer to the protein catalytic core by a distortion of the DNA in the central “4-base” region; (iv) cleavage occurs via a proposed two-metal mechanism, sometimes involving a unique “metal sharing” paradigm; (v) and finally, additional affinity and/or specificity contacts can arise from “adapted” scaffolds, in regions outside the core α/β fold. See Silva et al., 2011, Meganucleases and other tools for targeted genome engineering, Curr Gene Ther 11(1):11-27, incorporated by reference.

Some embodiments of the invention may utilize modified version of a nuclease. Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. Similar to the inactive dCas9 (RuvC- and HNH-), a Cas9 nickase is still able to bind DNA based on gRNA specificity, though nickases will only cut one of the DNA strands. The majority of CRISPR plasmids are derived from S. pyogenes and the RuvC domain can be inactivated by a D10A mutation and the HNH domain can be inactivated by an H840A mutation.

### Targeting Sequence

A nuclease may use the targeting specificity of a guide RNA (gRNA). As used herein, guide RNA and gRNA are used to mean any of gRNA, crRNA, tracrRNA, sgRNA, and others, where those RNAs are capable of guiding a Cas-type nuclease to a target. A gRNA is a species of targeting sequence. A CRISPR/Cas9 complex of the invention works optimally with a guide RNA that targets the viral genome. Guide RNA leads the CRISPR/Cas9 complex to the viral genome in order to cause viral genomic disruption. In an aspect of the invention, CRISPR/Cas9/gRNA complexes are designed to target specific viruses within a cell. It should be appreciated that any virus can be targeted using the composition of the invention. Identification of specific regions of the virus genome aids in development and designing of CRISPR/Cas9/gRNA complexes.

In an aspect of the invention, the CRISPR/Cas9/gRNA complexes are designed to target latent viruses within a cell. Once transfected within a cell, the CRISPR/Cas9/gRNA complexes cause repeated insertions or deletions to render the genome incapacitated, or due to number of insertions or deletions, the probability of repair is significantly reduced.

It will be appreciated that method and compositions of the invention can be used to target viral nucleic acid without interfering with host genetic material. Methods and compositions of the invention employ a targeting moiety such as a guide RNA that has a sequence that hybridizes to a target within the viral sequence. Methods and compositions of the invention may further use a targeted nuclease such as the cas9 enzyme, or a vector encoding such a nuclease, which uses the gRNA to bind exclusively to the viral genome and make double stranded cuts, thereby removing the viral sequence from the host.

Where the targeting moiety includes a guide RNA, the sequence for the gRNA, or the guide sequence, can be determined by examination of the viral sequence to find regions of about 20 nucleotides that are adjacent to a protospacer adjacent motif (PAM) and that do not also appear in the host genome adjacent to the protospacer motif.

Preferably a guide sequence that satisfies certain similarity criteria (e.g., at least 60% identical with identity biased toward regions closer to the PAM) so that a gRNA/cas9 complex made according to the guide sequence will bind to and digest specified features or targets in the viral sequence without interfering with the host genome. Preferably, the guide RNA corresponds to a nucleotide string next to a protospacer adjacent motif (PAM) (e.g., NGG, where N is any nucleotide) in the viral sequence. Preferably, the host genome lacks any region that (1) matches the nucleotide string according to a predetermined similarity criteria and (2) is also adjacent to the PAM. The predetermined similarity criteria may include, for example, a requirement of at least 12 matching nucleotides within 20 nucleotides 5′ to the PAM and may also include a requirement of at least 7 matching nucleotides within 10 nucleotides 5′ to the PAM. An annotated viral genome (e.g., from GenBank) may be used to identify features of the viral sequence and finding the nucleotide string next to a protospacer adjacent motif (PAM) in the viral sequence within a selected feature (e.g., a viral replication origin, a terminal repeat, a replication factor binding site, a promoter, a coding sequence, or a repetitive region) of the viral sequence. The viral sequence and the annotations may be obtained from a genome database.

Where multiple candidate gRNA targets are found in the viral genome, selection of the sequence to be the template for the guide RNA may favor the candidate target closest to, or at the 5′ most end of, a targeted feature as the guide sequence. The selection may preferentially favor sequences with neutral (e.g., 40% to 60%) GC content. Additional background regarding the RNA-directed targeting by endonuclease is discussed in U.S. Pub. 2015/0050699; U.S. Pub. 20140356958; U.S. Pub. 2014/0349400; U.S. Pub. 2014/0342457; U.S. Pub. 2014/0295556; and U.S. Pub. 2014/0273037, the contents of each of which are incorporated by reference for all purposes. Due to the existence of human genomes background in the infected cells, a set of steps are provided to ensure high efficiency against the viral genome and low off-target effect on the human genome. Those steps may include (1) target selection within viral genome, (2) avoiding PAM+target sequence in host genome, (3) methodologically selecting viral target that is conserved across strains, (4) selecting target with appropriate GC content, (5) control of nuclease expression in cells, (6) vector design, (7) validation assay, others and various combinations thereof. A targeting moiety (such as a guide RNA) preferably binds to targets within certain categories such as (i) latency related targets, (ii) infection and symptom related targets, and (iii) structure related targets.

A first category of targets for gRNA includes latency-related targets. The viral genome requires certain features in order to maintain the latency. These features include, but not limited to, master transcription regulators, latency-specific promoters, signaling proteins communicating with the host cells, etc. If the host cells are dividing during latency, the viral genome requires a replication system to maintain genome copy level. Viral replication origin, terminal repeats, and replication factors binding to the replication origin are good targets. Once those features are disrupted, the viruses may reactivate, which can be treated by conventional antiviral therapies.

A second category of targets for gRNA includes infection-related and symptom-related targets. Virus produces various molecules to facilitate infection. Once gained entrance to the host cells, the virus may start lytic cycle, which can cause cell death and tissue damage (HBV). In certain cases, such as HPV16, cell products (E6 and E7 proteins) can transform the host cells and cause cancers. Disrupting the key genome sequences (promoters, coding sequences, etc) can prevent further infection, and/or relieve symptoms, if not curing the disease.

A third category of targets for gRNA includes structure-related targets. Viral genome may contain repetitive regions to support genome integration, replication, or other functions. Targeting repetitive regions can break the viral genome into multiple pieces, which physically destroys the genome.

Where the nuclease is a cas protein, the targeting moiety is a guide RNA. Each cas protein requires a specific PAM next to the targeted sequence (not in the guide RNA). This is the same as for human genome editing. The current understanding the guide RNA/nuclease complex binds to PAM first, then searches for homology between guide RNA and target genome. Sternberg et al., 2014, DNA interrogation by the CRISPR RNA-guided endonuclease Cas9, Nature 507(7490):62-67. Once recognized, the DNA is digested 3-nt upstream of PAM. These results suggest that off-target digestion requires PAM in the host DNA, as well as high affinity between guide RNA and host genome right before PAM.

It may be preferable to use a targeting moiety that targets portions of the viral genome that are highly conserved. Viral genomes are much more variable than human genomes. In order to target different strains, the guide RNA will preferably target conserved regions. As PAM is important to sequence recognition, it is also essential to have PAM in the conserved region.

In a preferred embodiment, methods of the invention are used to deliver a nucleic acid to cells. The nucleic acid delivered to the cells may include a gRNA having the determined guide sequence or the nucleic acid may include a vector, such as a plasmid, that encodes an enzyme that will act against the target genetic material. Expression of that enzyme allows it to degrade or otherwise interfere with the target genetic material. The enzyme may be a nuclease such as the Cas9 endonuclease and the nucleic acid may also encode one or more gRNA having the determined guide sequence.

The gRNA targets the nuclease to the target genetic material. Where the target genetic material includes the genome of a virus, gRNAs complementary to parts of that genome can guide the degredation of that genome by the nuclease, thereby preventing any further replication or even removing any intact viral genome from the cells entirely. By these means, latent viral infections can be targeted for eradication.

The host cells may grow at different rate, based on the specific cell type. High nuclease expression is necessary for fast replicating cells, whereas low expression help avoiding off-target cutting in non-infected cells. Control of nuclease expression can be achieved through several aspects. If the nuclease is expressed from a vector, having the viral replication origin in the vector can increase the vector copy number dramatically, only in the infected cells. Each promoter has different activities in different tissues. Gene transcription can be tuned by choosing different promoters. Transcript and protein stability can also be tuned by incorporating stabilizing or destabilizing (ubiquitin targeting sequence, etc) motif into the sequence.

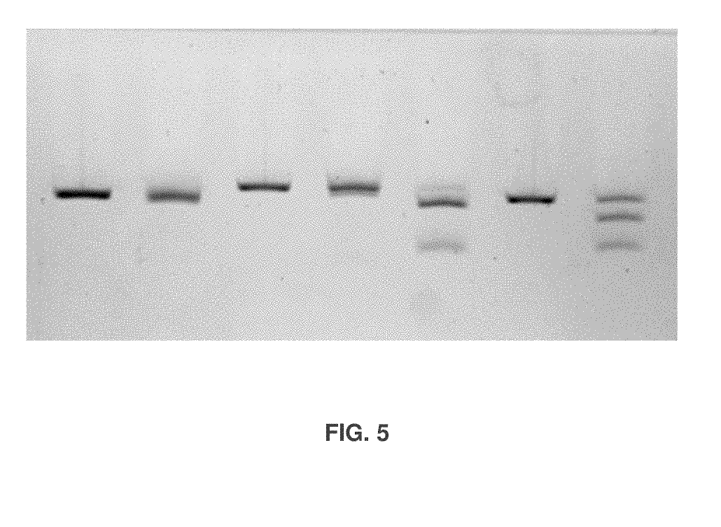

Specific promoters may be used for the gRNA sequence, the nuclease (e.g., cas9), other elements, or combinations thereof. For example, in some embodiments, the gRNA is driven by a U6 promoter. A vector may be designed that includes a promoter for protein expression (e.g., using a promoter as described in the vector sold under the trademark PMAXCLONING by Lonza Group Ltd (Basel, Switzerland). A vector may be a plasmid (e.g., created by synthesis instrument 255 and recombinant DNA lab equipment). In certain embodiments, the plasmid includes a U6 promoter driven gRNA or chimeric guide RNA (sgRNA) and a ubiquitous promoter-driven cas9. Optionally, the vector may include a marker such as EGFP fused after the cas9 protein to allow for later selection of cas9+cells. It is recognized that cas9 can use a gRNA (similar to the CRISPR RNA (crRNA) of the original bacterial system) with a complementary trans-activating crRNA (tracrRNA) to target viral sequences complementary to the gRNA. It has also been shown that cas9 can be programmed with a single RNA molecule, a chimera of the gRNA and tracrRNA. The single guide RNA (sgRNA) can be encoded in a plasmid and transcription of the sgRNA can provide the programming of cas9 and the function of the tracrRNA. See Jinek, 2012, A programmable dual-RNA-guided DNA endonuclease in adaptive bacterial immunity, Science 337:816-821 and especially FIG. 5A therein for background.

Using the above principles, methods and compositions of the invention may be used to target viral nucleic acid in an infected host without adversely influencing the host genome.

For additional background see Hsu, 2013, DNA targeting specificity of RNA-guided Cas9 nucleases, Nature Biotechnology 31(9):827-832; and Jinek, 2012, A programmable dual-RNA-guided DNA endonuclease in adaptive bacterial immunity, Science 337:816-821, the contents of each of which are incorporated by reference. Since the targeted locations are selected to be within certain categories such as (i) latency related targets, (ii) infection and symptom related targets, or (iii) structure related targets, cleavage of those sequences inactivates the virus and removes it from the host. Since the targeting RNA (the gRNA or sgRNA) is designed to satisfy according to similarity criteria that matches the target in the viral genetic sequence without any off-target matching the host genome, the latent viral genetic material is removed from the host without any interference with the host genome.

As an example, the Epstein-Barr virus (EBV), also called human herpesvirus 4 (HHV-4), was inactivated in cells using a CRISPR/Cas9/gRNA complex. EBV is a virus of the herpes family, and is one of the most common viruses in humans. The virus is approximately 122 nm to 180 nm in diameter and is composed of a double helix of DNA wrapped in a protein capsid. In this example, the Raji cell line serves as an appropriate in vitro model. The Raji cell line is the first continuous human cell line from hematopoietic origin and cell lines produce an unusual strain of Epstein-Barr virus while being one of the most extensively studied EBV models. To target the EBV genomes in the Raji cells, a Cas9 with specificity for EBV is needed.

The design of EBV-targeting CRISPR/Cas9 plasmids consisting of a U6 promoter driven chimeric guide RNA (sgRNA) and a ubiquitous promoter driven Cas9 that were obtained from Addgene, Inc. Commercially available guide RNAs and Cas9 nucleases may be used with the present invention. The EGFP marker fused after the Cas9 protein allowed selection of Cas9-positive cells.

Preferably guide RNAs are designed, whether or not commercially purchased, to target a specific part of an HPV genome. The target area in HPV is identified and guide RNA to target selected portions of the HPV genome are developed and incorporated into the composition of the invention. In an aspect of the invention, a reference genome of a particular strain of the virus is selected for guide RNA design.

In relation to EBV, for example, the reference genome from strain B95-8 was used as a design guide. Within a genome of interest, such as EBV, selected regions, or genes are targeted. For example, six regions can be targeted with seven guide RNA designs for different genome editing purposes. In relation to EBV, EBNA1 is the only nuclear Epstein-Barr virus (EBV) protein expressed in both latent and lytic modes of infection. While EBNA1 is known to play several important roles in latent infection, EBNA1 is crucial for many EBV functions including gene regulation and latent genome replication. Therefore, guide RNAs sgEBV4 and sgEBV5 were selected to target both ends of the EBNA1 coding region in order to excise this whole region of the genome. These “structural” targets enable systematic digestion of the EBV genome into smaller pieces. EBNA3C and LMP1 are essential for host cell transformation, and guide RNAs sgEBV3 and sgEBV7 were designed to target the 5′ exons of these two proteins respectively.

A nuclease such as a Cas-type nuclease cleaves nucleic acid of a virus infecting a cell and an inhibitor of DNA repair prevents the cleaved nucleic acid from being repaired.

### Inhibitor of DNA Repair

Methods of the invention include using one or more treatments to inhibit repair of the viral nucleic acid after it is cleaved by the nuclease. One or more type of inhibitor may be used. Exemplary types of inhibitors include: inhibitors of non-homologous end joining (NHEJ) repair or homologous recombination (HR); enzymes that modify free ends of nucleic acid; moieties such as nucleotide/nucleoside analogs that interfere with DNA synthesis; ligation of fragments with non-canonical 5′ or 3′ ends; others; or combinations thereof.

In preferred embodiments, the inhibitor includes a small molecule drug that inhibits NHEJ or HR. By suppressing or destroying those elements essential to end repair, the end repair processes are unable to operate and the resulting fragments remain unrepaired and degraded. The unrepaired and degraded fragments may promote apoptosis, induce cytotoxicity or may make the target nucleic acid more susceptible to other treatments that lead to apoptosis.

Proteins associated with non-homologous end repair include, but are not limited to: Ku80, Ku70, DNA-dependant protein kinase, catalytic subunits (DNA-PKcs), Artemis, Xrcc4, and Ligase IV, and non-homologous end repair can be inhibited by suppressing expression of those proteins. See, for example, Li, Y. H., Wang, X., Pan, Y., et al. (2012). Inhibition of non-homologous end joining repair impairs pancreatic cancer growth and enhances radiation response. PLoS One 7, e39588.; Srivastava, et al. (2012) An inhibitor of Nonhomologous End-Joining Abrogates Double Strand Break Repair and Impedes Cancer Progression,” incorporated by reference. Any enzyme, chemical or other small molecule that suppresses or inhibits those proteins or other elements essential to non-homologous end repair may be used as a treatment to prevent DNA repair. For example, Ligase IV has been found as a critical component in the sealing of double-strand breaks during non-homologous end joining. SCR7 inhibits expression of Ligase IV, thereby disrupting non-homologous end repair of fragmented nucleic acid. Inhibitors of DNA-PK cs include, for example, PI-3 Kinases, LY-294002, vanillin (Sigma), and NU-7026 (Valbiochem). Any suitable inhibitor of HR may be used. Typical inhibitors of HR will block an enzyme of a double-stranded break repair pathway such as ATM, ATR, MRN, RAD51 and paralogs, BRCA1, BRCA2, KU70/80, DNA-PKcs, Artemis, Ligase IV, or XRCC4. Suitable HR inhibitors may include mirin and caffeine. Specific ATR inhibitors (VE-821 and NU6027) have been identified based on cell-based screens and found to be especially toxic to cells deficient in p53. NU6027 also inhibits RAD51 focus formation (indicative of HR suppression). RI-1 covalently binds to the surface of RAD51, thereby reducing its focus formation. HR has also been targeted by inhibition of the ATM-CHK2 or ATR-CHK1 pathways. The selective ATM inhibitor KU55933 blocks ionization radiation (IR)-induced, ATM dependent phosphorylation and sensitized cancer cells to IR and topoisomerase inhibitors The nonspecific staurosporin analog UCN-01 is a potent CHK1 inhibitor. Small-molecule inhibitors of the human RecQ helicases include BLM (ML216) and WRN (NSC19630). An ERCC1 inhibitor, NSC130813, has been reported that synergizes the effect of cisplatin and mitomycin C. The PARP1 inhibitor olaparib shows promising results.

Any suitable inhibitor of NHEJ may be used. Genes and proteins associated with homologous end repair, include but are not limited to, Rad52, Rad51, ATM, BRAC1, BRAC2, MRN Complex, ATM, DNA, PK, ATR, and Blm. Homologous repair may be inhibited by suppressing expression of one or more of those proteins. Any enzyme, chemical or other component that negatively affects or suppresses or inhibits those proteins or other elements essential to homologous end repair may be used as a treatment to prevent DNA repair. In one example, 17-AAG (17-Allylmanio-17-Demethoxygeldanamycin) inhibits homologous end repair by causing degrading BRAC2 and altering the behavior of RAD51, which is critical for homologous end repair. NHEJ proteins such as the KU70/80 complex, Artemis, Ligase IV/XRCC4, Polm, and Poll may be targeted. One of the first inhibitors of DNA-PKcs was wortmannin. A derivative of quercetin, LY294002, has also been shown to possess similar properties. Recently, NU7026 has been reported to be a very selective and potent DNA-PK inhibitor. L189 is a potential Ligase IV inhibitor that blocks the activity of all three ligases, Ligase I, Ligase III, and Ligase IV. SCR7 has been identified as a potent inhibitor of end joining. For additional background, see Srivastava & Raghavan, 2015, DNA Double-strand break repair inhibitors as cancer therapies, Chem & Biol 22:17-29, incorporated by reference. Thus, the inhibitor may include small molecule such as KU55933; caffeine; VE-821; NU6027; UNC-01; mirin; RI-1; streptonigrin; RI-2; 3-ABA; olaparib; NU1025; NSC130813; wortmannin; NU7026; SCR7; or L189 to suppress homologous recombination (HR) or non-homologous end-joining (NHER).

An inhibitor may be an enzyme that modify free ends of nucleic acid, such as Antarctic phosphatase. Antarctic phosphatase catalyzes the removal of a 5′ phosphate group, rendering that free end unavailable for repair by end-joining or by template-dependent extension.

An inhibitor may include moieties such as nucleotide/nucleoside analogs that interfere with DNA synthesis. For example, where DNA repair would require template-dependent synthesis, a nucleotide analog may be taken up by a polymerase and arrest it from further activity.

Additionally or alternatively, repair may be inhibited by enhancing cell exonuclease activity, e.g. to increase degradation of SSB and DSB (single and double strand DNA breaks). For example, human exonuclease 1 (hEXO1) efficiently repairs DSB. hEXO1 is ubiquitinated and degraded in the proteasome. Thus, in some embodiments, a combination of a targeted endonuclease with a proteasome inhibitor are delivered to enhance hEXO-1 activity and synergize to kill viral DNA+cells.

In some embodiments, it is recognized that the double-stranded breaks (DSBs) introduced by Cas-type nuclease are primarily repaired via non-homologous end joining (NHEJ) and that DNA ligase IV (LIG4) is critical for NHEJ. Other LIGs (1-3) are involved in repair of SSB and DSB. Systems and methods described herein may include one or more small molecule inhibitors of LIG4 or other LIGs. For example, the compound L82 has been identified as an uncompetitive inhibitor of DNA ligase I. L67 is a compound that inhibits LIG1 and LIG3. Other compounds that have been identified as inhibitors of a DNA Ligase may be used.

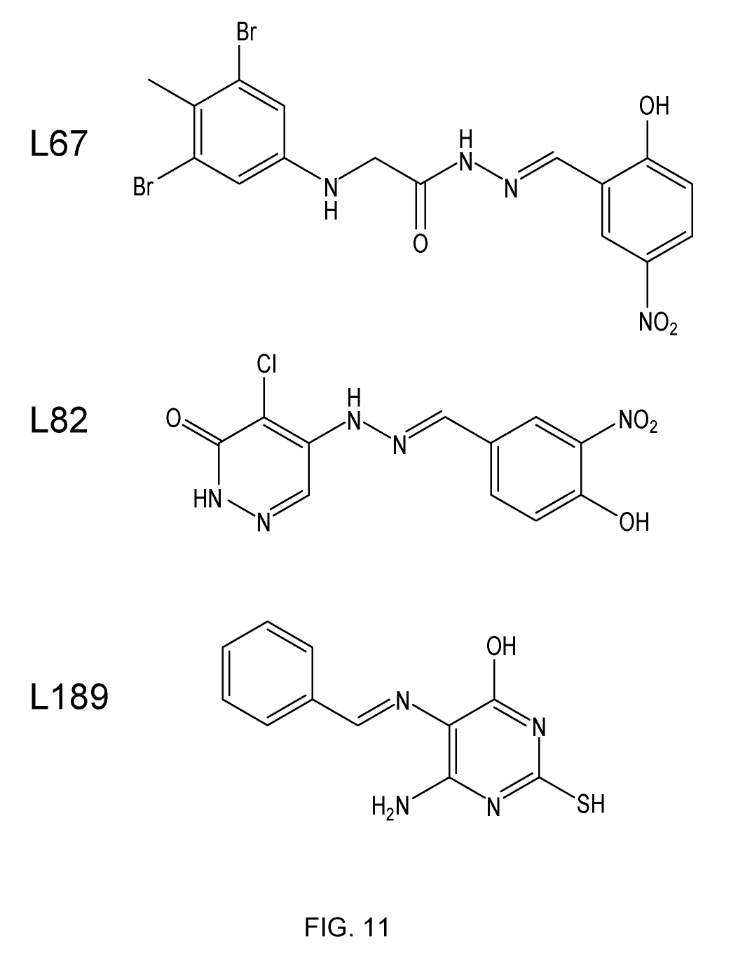

FIG. 11 shows three small molecule inhibitors of DNA ligase, L67, L82, and L189. L189 inhibits hLigI, hLigIIIβ, and hLigIV/XRCC4, L67 inhibits hLigI and hLigIIIβ, and L82 inhibits hLigI. Additional discussion may be found in Chen et al., 2008, Rational Design of Human DNA Ligase Inhibitors that Target Cellular DNA Replication and Repair, Cancer Res 68(9):3169-3177, incorporated by reference.

In some embodiments, the treated cell is provided with DNA fragments with non-canonical 5′ or 3′ ends. Those fragments may include sequence with at least partial homology to known, target viral sequences. The end-joining repair mechanisms (HR or NHEJ) may join those fragments to the free ends of the cut nucleic acid. In one embodiments, the fragments have 3′ ends that lack a 3′ hydroxyl group and thus present a di-deoxy 3′ end, which is not competent for further repair.

Additionally or alternatively, the inhibitor may be an enzyme that suppresses or destroys components (such as enzymes or proteins) necessary to end repair processes. Additionally or alternatively, the treatment may include any added molecule that would inhibit ligation of the resulting fragments after cleavage. For example, the treatment may be a chain terminator, which ligates to the fragments before the end repair operations.

An insight is to use gene-editing tools such as Cas9 along with an inhibitor of DNA repair, i.e., an inhibitor that would normally prevent successful gene-editing. By inhibiting end repair, ligation or both of the resulting fragments, the target nucleic acid (e.g. viral, mutated, or cancerous nucleic acid) is disrupted or destroyed. In some embodiments, leaving the target nucleic acid fragments unrepaired or non-ligated is enough to destroy the nucleic acid or treat the viral infection. In further embodiments, the unrepaired or non-ligated fragments may make the target nucleic acid more susceptible or sensitive to other therapies (including drug, chemical, radiation, etc.). For example, after the target nucleic acid fragments are exposed to the treatment and left unrepaired/non-ligated, methods of the invention provide for exposing the unrepaired/non-ligated fragments to another therapeutic agent to further destroy or degrade the target nucleic acid.

In certain embodiments, an inhibitor is used that prevents formation of a phosphodiester bond by, for example, inhibiting a polymerase or functioning as a chain terminator. For example, an enzyme or chemical may inhibit bond formation by removing or adding a phosphate group at the 5′ side or by removing the 3′ hydroxyl group to make a dideoxy end.

Inhibitor may include ddNTPs, nucleotides, nucleosides, or analogs thereof that prevent ligation or polymerase activity, when contacted and incorporated into a nucleic acid fragment. Such a moiety may repress viral reproduction by competing with natural dNTP/NTP substrates for incorporation into the nascent viral nucleic acid, thereby leading to chain termination. In certain embodiments, the chain terminator is a ddNTP. ddNTPs block polymerization and ligation when added to the end of a nucleic acid due to their lack of the 3′ hydroxyl group.

In certain embodiments, antiviral agents (such as nucleoside, nucleotide, and analogues thereof) may be used as the treatment. They compete with natural dNTP/NTP substrates for the incorporation into the nucleic acid thereby leading to chain termination or mutagenesis. See, Clerc and Neyts, Handb Exp Pharmacol. 2009;(189):53-84. doi: 10.1007/978-3-540-79086-0_3. For example, nucleoside analogues that possess a 3′ hydroxyl may act as a chain terminator, where the hydroxyl is conformationally constrained or sterically hindered from creating a phosphodiester linkage with incoming nucleotide. In such instance, chain elongation can be hampered by, for example, 2′-C-methyl or 4′azido nucleoside inhibitors of HCV replication. Canonical 3′deoxyribonucleotides have also been successfully used as a chain terminator. See, Shim et al. Antiviral Res. 2003 May;58(3):243-51. The following nucleosides, nucleotides, and analogues may be used as a chain terminator for purposes of the invention: Lamivudine triphosphate, Stavudine triphosphate, Zidoduvine triphosphate, Aciclovir triphosphate, Vidarabine triphosphate, Ribavirin triphosphate, 3TC (Lamivudine), d4T (Stavudine), AzT (Zidovudine), ara-A (Vidarabine), Aciclovir, Ribavirn, 3TCMP, d4TMP, AzTMP, ara-AMP, Aciclovir monophosphate, Ribavirin monophosphate, 3TCTP, d4TTP, AzTTP, ara-ATP, Aciclovir triphosphate, Ribavirin Triphosphate.

### Introduce to Cell

Methods of the invention include introducing into a host cell a nuclease and an inhibitor of DNA repair. The nuclease may be initially provided for delivery in any suitable form. For example, the nuclease may be delivered as an active enzyme or ribonucleoprotein (RNP) or the nuclease may be encoded in a nucleic acid, such as in a DNA vector or as mRNA Likewise, where the inhibitor is a protein, the inhibitor may initially be provided in any suitable form such as as a protein or encoded in a nucleic acid. Where the nuclease and the inhibitor are to be provided in a nucleic acid form, they may both be encoded on the same nucleic acid (e.g., DNA plasmid or mRNA) with or without a spacer or linker, or they may be separately delivered. In a preferred embodiment, the nuclease is obtained or delivered in a ribonucleoprotein (RNP) form, e.g. as a recombinant Cas9 protein duplexed with sgRNA or with crRNA+tracRNA, or as a recombinant TALEN protein. It may be found that delivery as RNP is more effective and less toxic than plasmid DNA, and that RNP permits delivery of pre-formed enzymatically active drug (which acts faster), and is only active in the cell for a very limited time (<24 hours), thus reducing non-specific toxicity and off-target activity. RNP can be directly electroporated into primary tissues, e.g. peripheral blood mononuclear cells (PBMCs), for ex vivo transplant indications. RNP, like mRNA or pDNA, can also be incorporated into cationic lipid nanoparticles for in vivo delivery indications, e.g. cancer.

The nuclease and the inhibitor may be delivered to the infected cells together (e.g., as part of a single composition) or they may be delivered separately, wholly or partially simultaneously or separately. Either or both of the nuclease and inhibitor may be provided with a pharmaceutically acceptable carrier or prepared for delivery orally, intravenously, topically, or by any suitable method. Either or both of the nuclease and the inhibitor may be delivered using a suitable viral or non-viral vector or delivery method or other suitable format. For example, the nuclease, targeting sequence, and the treatment may be delivered on the same vehicle, whether nucleic acid, plasmid, or viral vector. Alternatively, the nuclease and targeting sequence may be delivered in one manner, and the treatment may be delivered in a separate manner. For example, a cocktail may include: (i) a vector encoding the nuclease and the targeting sequence; and (ii) the treatment or a vector encoding the treatment.

In some embodiments, a Cas-type nuclease and inhibitor are introduced into a cell. A guide RNA is designed to target at least one category of sequences of the viral genome. In addition to latent infections this invention can also be used to control actively replicating viruses by targeting the viral genome before it is packaged or after it is ejected. In some embodiments, a cocktail of guide RNAs may be introduced into a cell along with the nuclease and inhibitor. The guide RNAs are designed to target numerous categories of sequences of the viral genome. By targeting several areas along the genome, the double strand break at multiple locations fragments the genome, lowering the possibility of repair. In some embodiments, several guide RNAs are added to create a cocktail to target different categories of sequences. For example, two, five, seven or eleven guide RNAs may be present in a CRISPR cocktail targeting three different categories of sequences. However, any number of gRNAs may be introduced into a cocktail to target categories of sequences. In preferred embodiments, the categories of sequences are important for genome structure, host cell transformation, and infection latency, respectively. A nuclease, inhibitor or both may be delivered into cells by any suitable method including viral vectors and non-viral vectors.

Viral vectors may include retroviruses, lentiviruses, adenoviruses, and adeno-associated viruses. It should be appreciated that any viral vector may be incorporated into the present invention to effectuate delivery of the complex into a cell. Some viral vectors may be more effective than others, depending on the complex designed for digestion or incapacitation. In an aspect of the invention, the vectors contain essential components such as origin of replication, which is necessary for the replication and maintenance of the vector in the host cell. Use of viral vectors as delivery vectors are known in the art. See for example U.S. Pub. 2009/0017543 to Wilkes et al., the contents of which are incorporated by reference. A viral vector may be provided by a retrovirus.

A retrovirus is a single-stranded RNA virus that stores its nucleic acid in the form of an mRNA genome (including the 5′ cap and 3′ PolyA tail) and targets a host cell as an obligate parasite. In some methods in the art, retroviruses have been used to introduce nucleic acids into a cell. Once inside the host cell cytoplasm the virus uses its own reverse transcriptase enzyme to produce DNA from its RNA genome. This new DNA is then incorporated into the host cell genome. Retroviral vectors can either be replication-competent or replication-defective. In some embodiments of the invention, retroviruses are incorporated to effectuate transfection into a cell, however the nuclease/gRNA complexes are designed to target the viral genome.

In some embodiments, lentiviruses (a subclass of retroviruses) are used as viral vectors. Lentiviruses can be adapted as delivery vehicles (vectors) given their ability to integrate into the genome of non-dividing cells, which is the unique feature of lentiviruses as other retroviruses can infect only dividing cells. The viral genome in the form of RNA is reverse-transcribed when the virus enters the cell to produce DNA, which is then inserted into the genome at a random position by the viral integrase enzyme. The vector, now called a provirus, remains in the genome and is passed on to the progeny of the cell when it divides.

As opposed to lentiviruses, adenoviral DNA does not integrate into the genome and is not replicated during cell division. Adenovirus and the related AAV may be used as delivery vectors since they do not integrate into the host's genome. In some aspects of the invention, only the viral genome to be targeted is effected by the nuclease/gRNA complexes, and not the host's cells. Adeno-associated virus (AAV) is a small virus that infects humans and some other primate species. AAV can infect both dividing and non-dividing cells and may incorporate its genome into that of the host cell. For example, because of its potential use as a gene therapy vector, researchers have created an altered AAV called self-complementary adeno-associated virus (scAAV). Whereas AAV packages a single strand of DNA and requires the process of second- strand synthesis, scAAV packages both strands which anneal together to form double stranded DNA. By skipping second strand synthesis scAAV allows for rapid expression in the cell. Otherwise, scAAV carries many characteristics of its AAV counterpart. Addtionally or alternatively, methods and compositions of the invention may use herpesvirus, poxvirus, alphavirus, or vaccinia virus as a means of delivery vectors.

In certain embodiments of the invention, non-viral vectors may be used to effectuate transfection.

Non-viral vectors for the delivery of nucleic acids and other moieties include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam and Lipofectin). Cationic and neutral lipids that are suitable for efficient receptor-recognition lipofection of polynucleotides include those described in U.S. Pat. No. 7,166,298 to Jessee or U.S. Pat. No. 6,890,554 to Jesse, the contents of each of which are incorporated by reference. Delivery can be to cells (e.g. in vitro or ex vivo administration) or target tissues (e.g. in vivo administration).

Non-viral vectors may include synthetic vectors based on cationic lipids or polymers which can complex with negatively charged nucleic acids to form particles with a diameter in the order of 100 nm. The complex protects nucleic acid from degradation by nuclease. The surfaces of the cationic non-viral vectors have properties that minimize interaction with blood components, reduce reticuloendothelial system uptake, decrease their toxicity and increase binding affinity with the target cells.

Non-viral vectors may be modified to effectuate targeted delivery and transfection. PEGylation (i.e. modifying the surface with polyethyleneglycol) is the predominant method used to reduce the opsonization and aggregation of non-viral vectors and minimize the clearance by reticuloendothelial system, leading to a prolonged circulation lifetime after intravenous (i.v.) administration. PEGylated nanoparticles are therefore often referred as “stealth” nanoparticles. The nanoparticles that are not rapidly cleared from the circulation will have a chance to encounter infected cells.

However, PEG on the surface can decrease the uptake by target cells and reduce the biological activity. Therefore, to attach targeting ligand to the distal end of the PEGylated component is necessary; the ligand is projected beyond the PEG “shield” to allow binding to receptors on the target cell surface. When cationic liposome is used as gene carrier, the application of neutral helper lipid is helpful for the release of nucleic acid, besides promoting hexagonal phase formation to enable endosomal escape. In some embodiments of the invention, neutral or anionic liposomes are developed for systemic delivery of nucleic acids and obtaining therapeutic effect in experimental animal model. Designing and synthesizing novel cationic lipids and polymers, and covalently or non-covalently binding gene with peptides, targeting ligands, polymers, or environmentally sensitive moieties also attract many attentions for resolving the problems encountered by non-viral vectors. The application of inorganic nanoparticles (for example, metallic nanoparticles, iron oxide, calcium phosphate, magnesium phosphate, manganese phosphate, double hydroxides, carbon nanotubes, and quantum dots) in delivery vectors can be prepared and surface-functionalized in many different ways.

In some embodiments, compositions may be conjugated to nano-systems for systemic therapy, such as liposomes, albumin-based particles, PEGylated proteins, biodegradable polymer-drug composites, polymeric micelles, dendrimers, among others. See Davis et al., 2008, Nanotherapeutic particles: an emerging treatment modality for cancer, Nat Rev Drug Discov. 7(9):771-782, incorporated by reference. Long circulating macromolecular carriers such as liposomes, can exploit the enhanced permeability and retention effect for preferential extravasation from tumor vessels. In certain embodiments, compositions of the invention are conjugated to or encapsulated into a liposome or polymerosome for delivery to a cell. For example, liposomal anthracyclines have achieved highly efficient encapsulation, and include versions with greatly prolonged circulation such as liposomal daunorubicin and pegylated liposomal doxorubicin. See Krishna et al., Carboxymethylcellulose-sodium based transdermal drug delivery system for propranolol, J Pharm Pharmacol. 1996 Apr; 48(4):367-70.

Liposomes and polymerosomes can contain a plurality of solutions and compounds. In certain embodiments, compositions of the invention are coupled to or encapsulated in polymersomes. As a class of artificial vesicles, polymersomes are tiny hollow spheres that enclose a solution, made using amphiphilic synthetic block copolymers to form the vesicle membrane. Common polymersomes contain an aqueous solution in their core and are useful for encapsulating and protecting the nuclease, the inhibitor, nucleic acids encoding the nuclease or inhibitor, or combinations thereof. The polymersome membrane provides a physical barrier that isolates the encapsulated material from external materials, such as those found in biological systems. Polymerosomes can be generated from double emulsions by known techniques, see Lorenceau et al., 2005, Generation of Polymerosomes from Double-Emulsions, Langmuir 21(20):9183-6, incorporated by reference.

Aspects of the invention provide for numerous uses of delivery vectors. Selection of the delivery vector is based upon the cell or tissue targeted and the specific nuclease or inhibitor. For example, in the EBV example discussed above, since lymphocytes are known for being resistant to lipofection, nucleofection (a combination of electrical parameters generated by a device called Nucleofector, with cell-type specific reagents to transfer a substrate directly into the cell nucleus and the cytoplasm) was necessitated for DNA delivery into the Raji cells. The Lonza pmax promoter drives Cas9 expression as it offered strong expression within Raji cells. 24 hours after nucleofection, obvious EGFP signals were observed from a small proportion of cells through fluorescent microscopy. The EGFP-positive cell population decreased dramatically, however, <10% transfection efficiency 48 hours after nucleofection was measured.

Any suitable delivery system or pathway may be used for the nuclease, the inhibitor, or both. Suitable pathways include transdermal, transmucal, nasal, ocular and pulmonary routes. Drug delivery systems may include liposomes, proliposomes, microspheres, gels, prodrugs, cyclodextrins, etc. Aspects of the invention utilize nanoparticles composed of biodegradable polymers to be transferred into an aerosol for targeting of specific sites or cell populations in the lung, providing for the release of the drug in a predetermined manner and degradation within an acceptable period of time. Controlled-release technology (CRT), such as transdermal and transmucosal controlled-release delivery systems, nasal and buccal aerosol sprays, drug-impregnated lozenges, encapsulated cells, oral soft gels, iontophoretic devices to administer drugs through skin, and a variety of programmable, implanted drug-delivery devices are used in conjunction with the complexes of the invention of accomplishing targeted and controlled delivery. In some embodiments, the invention provides a composition for topical application (e.g., in vivo, directly to skin of a person). The composition may be applied superficially (e.g., topically). The composition provides a nuclease or gene therefore and includes a pharmaceutically acceptable diluent, adjuvant, or carrier. Preferably, a carrier used in accordance with the subject invention is approved for animal or human use by a competent governmental agency, such as the US Food and Drug Administration (FDA) or the like. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. These formulations contain from about 0.01% to about 100%, preferably from about 0.01% to about 90% of the MFB extract, the balance (from about 0% to about 99.99%, preferably from about 10% to about 99.99% of an acceptable carrier or other excipients. A more preferred formulation contains up to about 10% MFB extract and about 90% or more of the carrier or excipient, whereas a typical and most preferred composition contains about 5% MFB extract and about 95% of the carrier or other excipients. Formulations are described in a number of sources that are well known and readily available to those skilled in the art.

### Mechanism of Action

Once inside the cell, the nuclease cuts viral nucleic acid and the inhibitor prevents repair of the cut nucleic acid.

Preferably, the nuclease is specifically targeted to viral genomes, e.g., by the sequence of ZFN or by a gRNA with a Cas9. The nuclease cuts the viral nucleic acid and the inhibitor prevents repair. In some embodiments, methods and compositions of the invention use a nuclease such as Cas9 to target latent viral genomes, thereby reducing the chances of proliferation.

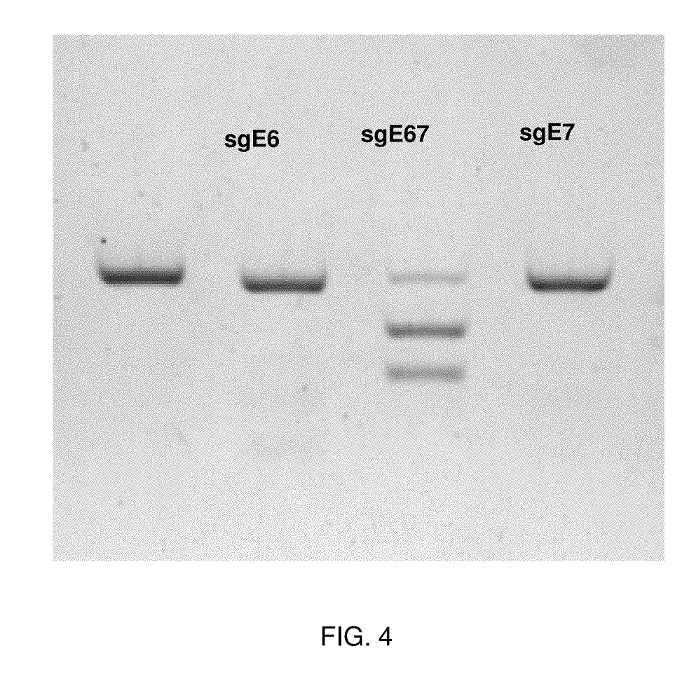

The following describes using Cas9 endonuclease and gRNA for targeted cutting of the HPV genome. It is understood that this description is applicable to other nucleases. FIG. 4 shows the results of successfully cleaving the HPV genome using Cas9 endonuclease, a gRNA for E6, and a gRNA for E7. The nuclease forms a complex with the gRNA (e.g., crRNA+tracrRNA or sgRNA). The complex cuts the viral nucleic acid in a targeted fashion to incapacitate the viral genome. The Cas9 endonuclease causes a double strand break in the viral genome. By targeted several locations along the viral genome and causing not a single strand break, but a double strand break, the genome is effectively cut a several locations along the genome.

The inhibitor prevents repair of the double stranded breaks. In some embodiments, an inhibitor such as a small molecule drug prevents homologous or non-homologous end repair. In certain embodiments, the inhibitor suppresses or destroys elements (such as enzymes or proteins) necessary to end repair processes. Additionally or alternatively, the inhibitor may otherwise prevent ligation of the resulting fragments after cleavage. For example, the treatment may be a chain terminator, which ligates to the fragments before the end repair operations. The use of terminators is in contrast to targeted nuclease schemes that rely on end repair mechanisms (e.g. cleave mutated sequence between fragments and re-ligate fragments) to create a desired change within the genomic region of interest, such as altering its expression. By inhibiting end repair, ligation or both of the resulting fragments, the target nucleic acid (e.g. viral, mutated, or cancerous nucleic acid) is disrupted or destroyed. In some embodiments, the target nucleic acid destruction itself may be enough to treat the infection. In further embodiments, the unrepaired or non-ligated fragments may make the target nucleic acid more susceptible or sensitive to other therapies (including drug, chemical, radiation, etc.)

In preferred embodiments, compositions and methods of the invention are used to treat latent viral infections. Viruses known or suspected to exhibit a latency phase include viruses of the Herpesviridae family (e.g., Herpes simplex virus-1 (HSV-1), Herpes simplex virus-2 (HSV-2), Varicella zoster virus (VZV), Epstein-Barr virus (EBV), Cytomegalovirus (CMV), Roseolovirus, Herpes lymphotropic virus, Kaposi's sarcoma-associated herpesvirus (KSHV)) among others (e.g., pseuodrabies virus). Latency is distinguished from lytic infection; in lytic infection many Herpes virus particles are produced and then burst or lyse the host cell. Lytic infection is sometimes known as “productive” infection. Latent cells harbor the virus for long time periods, then occasionally convert to productive infection which may lead to a recurrence of symptomatic Herpes symptoms. During latency, most of the Herpes DNA is inactive, with the exception of LAT, which accumulates within infected cells. Treating a latent viral infection with a targeted nuclease and a treatment that prevents DNA repair may be particularly beneficial in preventing any recurrence of a productive infection.

After a treatment is used to inhibit end repair, one or more therapeutics may be applied to further degrade target nucleic acid or induce apoptosis in the diseased or infected cell. The therapeutic may include, for example, application of radiation therapy, application of pharmaceuticals, antibiotics, or other chemical compounds, or a combination thereof. In a preferred embodiment, a treatment that prevents DNA repair includes an exonuclease. In one example, chemotherapy or cytotoxic drugs may be applied after inhibition of end repair for further treatment. Suitable chemotherapy drugs include alkylating agents, antimetabolites, anthracyclines and other anti-tumor antibiotics, topoisomerase inhibitors, and miotic inhibitors, corticosteroids. In another example, hydroxamic acid-based compounds, such as trichostatin A (TSA), can be used to induce cytoxicity or further apoptosis of cells with the degraded nucleic acid (i.e. nucleic acid fragments with end repair inhibited).

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

### Example 1

### Digesting Viral Nucleic Acid I

Methods and materials of the present invention may be used to digest foreign nucleic acid such as a genome of a hepatitis B virus (HBV).

It may be preferable to receive annotations for the HBV genome (i.e., that identify important features of the genome) and choose a candidate for targeting by enzymatic degredation that lies within one of those features, such as a viral replication origin, a terminal repeat, a replication factor binding site, a promoter, a coding sequence, and a repetitive region.

The use of Cas9 may be validated using an in vitro assay. To demonstrate, an in vitro assay is performed with cas9 protein and DNA amplicons flanking the target regions. Here, the target is amplified and the amplicons are incubated with cas9 and a gRNA having the selected nucleotide sequence for targeting. As shown in FIG. 14, DNA electrophoresis shows strong digestion at the target sites.

FIG. 5 shows a gel resulting from an in vitro CRISPR assay against HBV. Lanes 1, 3, and 6: PCR amplicons of HBV genome flanking RT, Hbx-Core, and PreS1. Lane 2, 4, 5, and 7: PCR amplicons treated with sgHBV-RT, sgHBV-Hbx, sgHBV-Core, sgHBV-PreS1. The presence of multiple fragments especially visible in lanes 5 and 7 show that sgHBV-Core and sgHBV-PreS1 provide especially attractive targets in the context of HBV and that use of systems and methods of the invention may be shown to be effective by an in vitro validation assay.

FIG. 5 gives results of digesting foreign nucleic acid. The nuclease forms a complex with the gRNA (e.g., crRNA+tracrRNA or sgRNA). The complex cuts the viral nucleic acid in a targeted fashion to incapacitate the viral genome. The Cas9 endonuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even with repair mechanisms, the genome is render incapacitated.

### Example 2

### Digesting Viral Nucleic Acid II

An exemplary assay shows the digestion of viral nucleic acid. Burkitt's lymphoma cell lines Raji, Namalwa, and DG-75 were obtained from ATCC and cultured in RPMI 1640 supplemented with 10% FBS and PSA, following ATCC recommendation. Human primary lung fibroblast IMR-90 was obtained from Coriell and cultured in Advanced DMEM/F-12 supplemented with 10% FBS and PSA.

Plasmids consisting of a U6 promoter driven chimeric guide RNA (sgRNA) and a ubiquitous promoter driven Cas9 were obtained from Addgene, as described by Cong L et al. (2013) Multiplex Genome Engineering Using CRISPR/Cas Systems. Science 339:819-823.

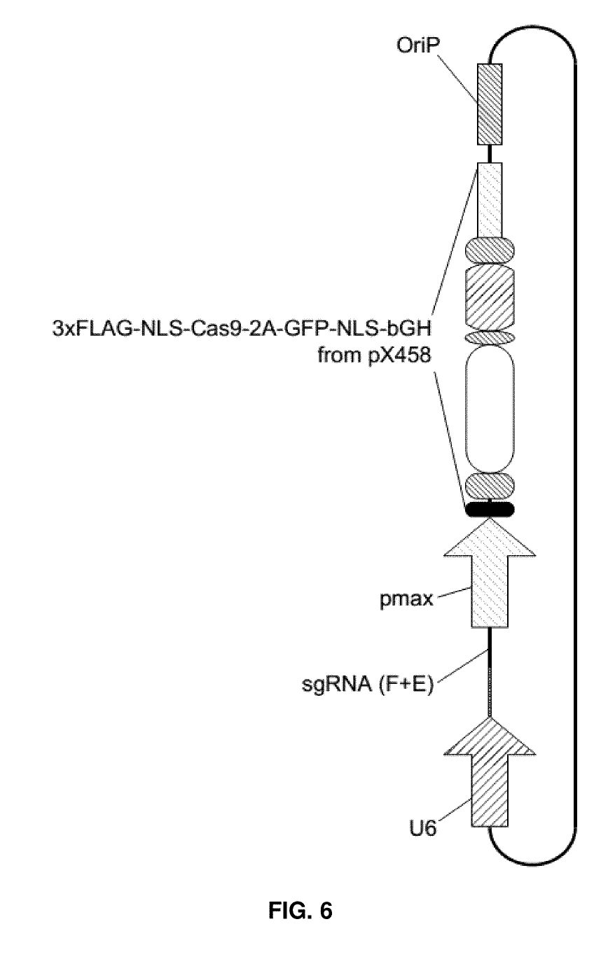

FIG. 6 shows a plasmid according to certain embodiments. An EGFP marker fused after the Cas9 protein allowed selection of Cas9-positive cells. A modified chimeric guide RNA stem-loop design was adapted for more efficient Pol-III transcription and more stable stem-loop structure (Chen B et al. (2013) Dynamic Imaging of Genomic Loci in Living Human Cells by an Optimized CRISPR/Cas System. Cell 155:1479-1491).

We obtained pX458 from Addgene, Inc. A modified CMV promoter with a synthetic intron (pmax) was PCR amplified from Lonza control plasmid pmax-GFP. A modified guide RNA sgRNA(F+E) was ordered from IDT. EBV replication origin oriP was PCR amplified from B95-8 transformed lymphoblastoid cell line GM12891. We used standard cloning protocols to clone pmax, sgRNA(F+E) and oriP to pX458, to replace the original CAG promoter, sgRNA and fl origin. We designed EBV sgRNA based on the B95-8 reference, and ordered DNA oligos from IDT. The original sgRNA place holder in pX458 serves as the negative control.

Lymphocytes are known for being resistant to lipofection, and therefore we used nucleofection for DNA delivery into Raji cells. We chose the Lonza pmax promoter to drive Cas9 expression as it offered strong expression within Raji cells. We used the Lonza Nucleofector II for DNA delivery. 5 million Raji or DG-75 cells were transfected with 5 ug plasmids in each 100-ul reaction. Cell line Kit V and program M-013 were used following Lonza recommendation. For IMR-90, 1 million cells were transfected with 5 ug plasmids in 100 ul Solution V, with program T-030 or X-005. 24 hours after nucleofection, we observed obvious EGFP signals from a small proportion of cells through fluorescent microscopy. The EGFP-positive cell population decreased dramatically after that, however, and we measured <10% transfection efficiency 48 hours after nucleofection. We attributed this transfection efficiency decrease to the plasmid dilution with cell division. To actively maintain the plasmid level within the host cells, we redesigned the CRISPR plasmid to include the EBV origin of replication sequence, oriP. With plasmid replication in the cells, the transfection efficiency rose to >60%.

To design guide RNA targeting the EBV genome, we relied on the EBV reference genome from strain B95-8.

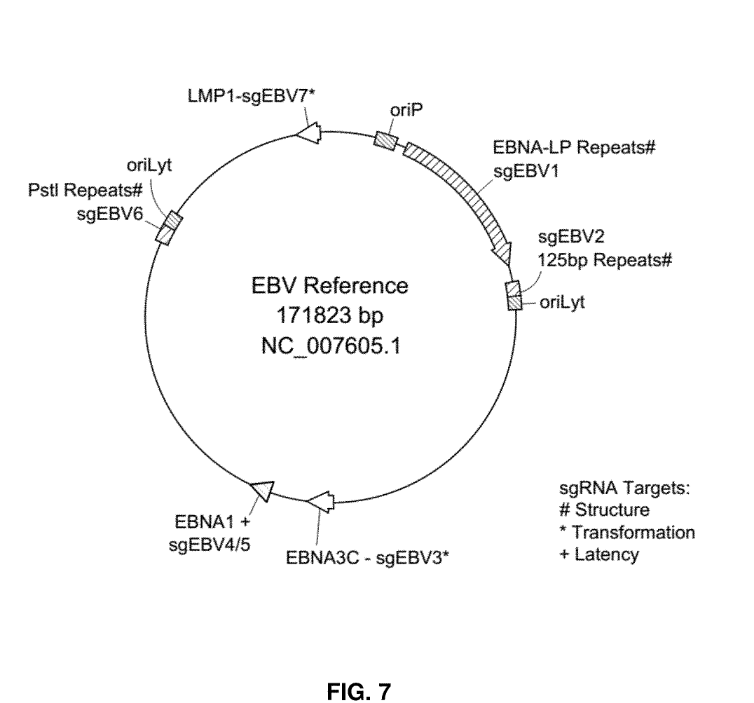

FIG. 7 diagrams the EBV genome. We targeted six regions with seven guide RNA designs for different genome editing purposes. The guide RNAs are listed in Table S1 in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

EBNA1 is crucial for many EBV functions including gene regulation and latent genome replication. We targeted guide RNA sgEBV4 and sgEBV5 to both ends of the EBNA1 coding region in order to excise this whole region of the genome. Guide RNAs sgEBV1, 2 and 6 fall in repeat regions, so that the success rate of at least one CRISPR cut is multiplied. These “structural” targets enable systematic digestion of the EBV genome into smaller pieces. EBNA3C and LMP1 are essential for host cell transformation, and we designed guide RNAs sgEBV3 and sgEBV7 to target the 5′ exons of these two proteins respectively.

### EBV Genome Editing.

The double-strand DNA breaks generated by CRISPR are repaired with small deletions. These deletions will disrupt the protein coding and hence create knockout effects. SURVEYOR assays confirmed efficient editing of individual sites. Beyond the independent small deletions induced by each guide RNA, large deletions between targeting sites can systematically destroy the EBV genome.

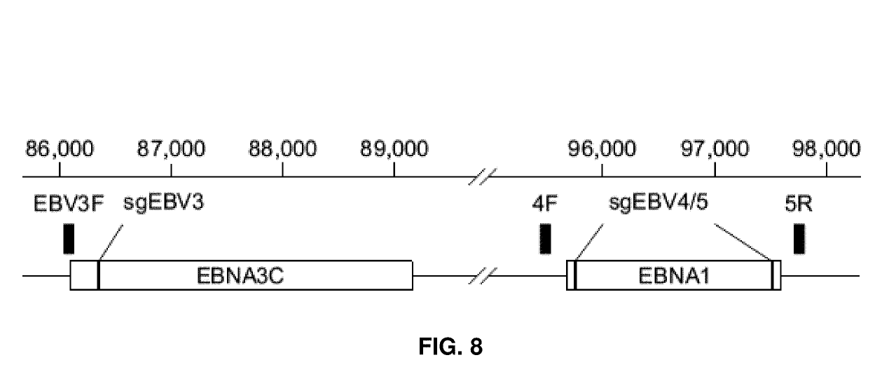

FIG. 8 shows genomic context around guide RNA sgEBV2 and PCR primer locations.

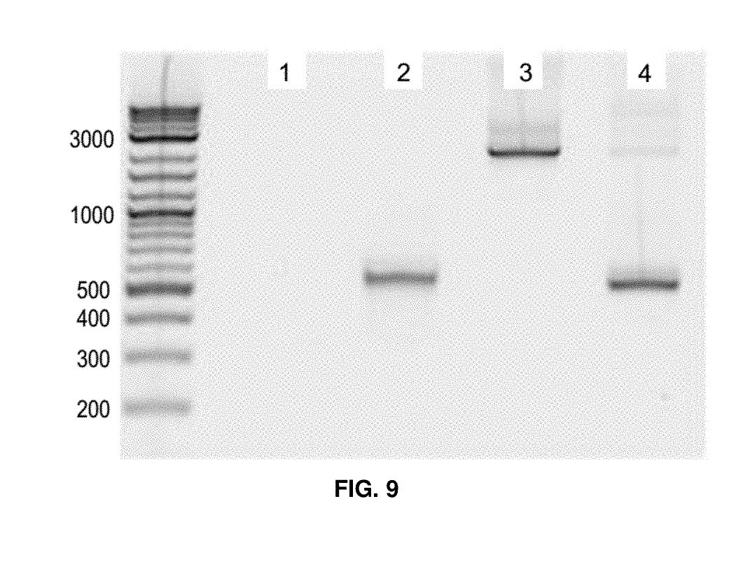

FIG. 9 shows a large deletion induced by sgEBV2, where lane 1-3 are before, 5 days after, and 7 days after sgEBV2 treatment, respectively. Guide RNA sgEBV2 targets a region with twelve 125-bp repeat units (FIG. 8). PCR amplicon of the whole repeat region gave a ˜1.8-kb band (FIG. 7). After 5 or 7 days of sgEBV2 transfection, we obtained ˜0.4-kb bands from the same PCR amplification (FIG. 7). The ˜1.4-kb deletion is the expected product of repair ligation between cuts in the first and the last repeat unit (FIG. 6).

DNA sequences flanking sgRNA targets were PCR amplified with Phusion DNA polymerase. SURVEYOR assays were performed following manufacturer's instruction. DNA amplicons with large deletions were TOPO cloned and single colonies were used for Sanger sequencing. EBV load was measured with Taqman digital PCR on Fluidigm BioMark. A Taqman assay targeting a conserved human locus was used for human DNA normalization. 1 ng of single-cell whole-genome amplification products from Fluidigm C1 were used for EBV quantitative PCR. We further demonstrated that it is possible to delete regions between unique targets. Six days after sgEBV4-5 transfection, PCR amplification of the whole flanking region (with primers EBV4F and 5R) returned a shorter amplicon, together with a much fainter band of the expected 2 kb (FIG. 9).

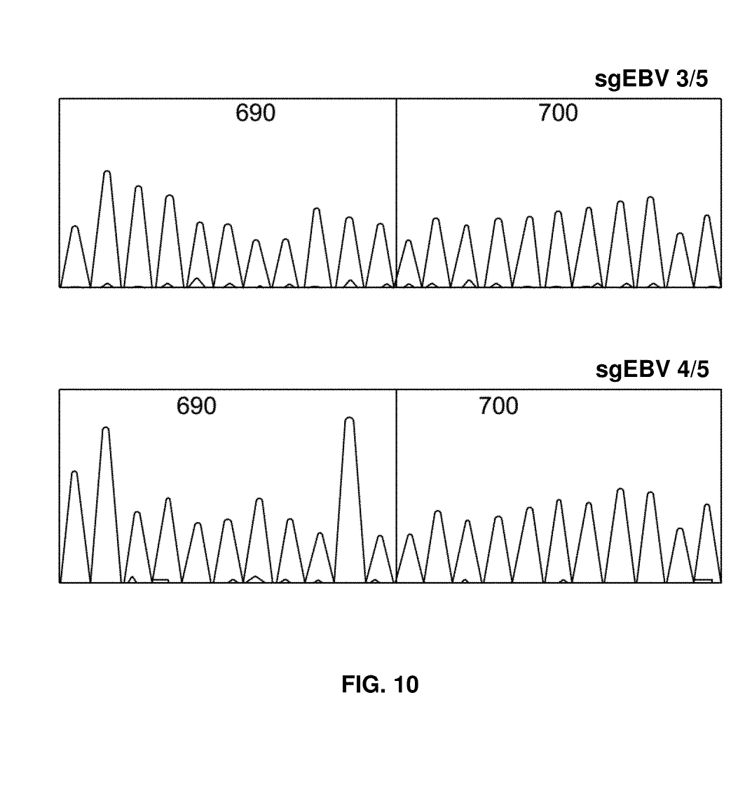

FIG. 10 shows that Sanger sequencing of amplicon clones confirmed the direct connection of the two expected cutting sites. A similar experiment with sgEBV3-5 also returned an even larger deletion, from EBNA3C to EBNA1. Additional information such as primer design is shown in Wang and Quake, 2014, RNA-guided endonuclease provides a therapeutic strategy to cure latent herpesviridae infection, PNAS 111(36):13157-13162 and in the Supporting Information to that article published online at the PNAS website, and the contents of both of those documents are incorporated by reference for all purposes.

### Targets For EBV Treatment.

The seven guide RNAs in our CRISPR cocktail target three different categories of sequences which are important for EBV genome structure, host cell transformation, and infection latency, respectively. To understand the most essential targets for effective EBV treatment, we transfected Raji cells with subsets of guide RNAs. Although sgEBV4/5 reduced the EBV genome by 85%, they could not suppress cell proliferation as effectively as the full cocktail. Guide RNAs targeting the structural sequences (sgEBV1/2/6) could stop cell proliferation completely, despite not eliminating the full EBV load (26% decrease). We conclude that systematic destruction of EBV genome structure appears to be more effective than targeting specific key proteins for EBV treatment.

What is claimed is:

1. A system for targeted treatment of a viral infection, the system comprising:
a nuclease capable of cutting viral nucleic acid into fragments;
a targeting sequence that targets the nuclease to the viral nucleic acid; and
a DNA repair inhibitor.

2. The system of claim 1, wherein the DNA repair inhibitor is a molecule that prevents end-joining.

3. The system of claim 1, wherein the DNA repair inhibitor is selected from the group consisting of a chain-terminating nucleotide, chain-terminating nucleotide analogue, a chain-terminating nucleoside, a chain-terminating nucleoside analogue, and a phosphatase.

4. The system of claim 3, wherein the chain-terminating nucleotide is a dideoxynucleotide.

5. The system of claim 1, wherein the nuclease is selected from the group consisting of a zinc-finger nuclease, a transcription activator-like effector nuclease, a meganuclease, and a Cas9 endonuclease.

6. The system of claim 1, wherein the targeting sequence comprises one or more guide RNAs.

7. The system of claim 1, wherein the viral nucleic acid is from a virus selected from the group consisting of Adenovirus, Herpes simplex, type 1, Herpes simplex, type 2, Varicella-zoster virus, Epstein-barr virus, Human cytomegalovirus, Human herpesvirus, type 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, Human immunodeficiency virus (HIV), Influenza virus, Guanarito virus, Junin virus, Lassa virus, Machupo virus, Sabiá virus, Crimean-Congo hemorrhagic fever virus, Ebola virus, Marburg virus, Measles virus, Mumps virus, Parainfluenza virus, Respiratory syncytial virus, Human metapneumovirus, Hendra virus, Nipah virus, Rabies virus, Hepatitis D, Rotavirus, Orbivirus, Coltivirus, Banna virus, and Merkel cell polyomavirus.

8. The system of claim 1, wherein the nuclease and the targeting sequence are introduced in a vector.

9. The system of claim 8, wherein the vector further comprises the DNA repair inhibitor.

10. The system of claim 8, wherein the vector is a viral vector.

11. The system of claim 10, wherein the viral vector is selected from the group consisting of retrovirus, lentivirus, adenovirus, herpes virus, pox virus, alpha virus, vaccina virus, adeno-associated viruses, hepatitis B virus, human papillomavirus, and chimeric viral vectors.

12. The system of claim 8, wherein the vector further comprises a member selected from the group consisting of a plasmid, a nanoparticle, a cationic lipid, a cationic polymer, a metallic nanopolymer, a nanorod, a liposome, a micelle, a microbubble, a cell-penetrating peptide, and a liposphere.

13. A composition for targeted treatment of nucleic acid, the composition comprising:
a vector encoding a nuclease that cuts target nucleic acid into fragments and a targeting sequence that targets the nuclease to the target nucleic acid; and
a DNA repair inhibitor.

14. The composition of claim 13, wherein the DNA repair inhibitor inhibits end-joining.

15. The composition of claim 13, wherein the treatment is selected from a chain-terminating nucleotide, chain-terminating nucleotide analogue, a chain-terminating nucleoside, a chain-terminating nucleoside analogue, and a phosphatase.

16. The composition of claim 14, wherein the treatment comprises a dideoxynucleotide.

17. The composition of claim 13, wherein the nuclease is selected from the group consisting of a zinc-finger nuclease, a transcription activator-like effector nuclease, a meganuclease, and a Cas9 endonuclease.

18. The composition of claim 13, wherein the target nucleic acid is from a virus.

19. The composition of claim 18, wherein the virus is selected from the group consisting of Adenovirus, Herpes simplex, type 1, Herpes simplex, type 2, Varicella-zoster virus, Epstein-barr virus, Human cytomegalovirus, Human herpesvirus 8, Human papillomavirus, BK virus, JC virus, Smallpox, Hepatitis B virus, Human bocavirus, Parvovirus B19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, Human immunodeficiency virus (HIV), Influenza virus, Guanarito virus, Junin virus, Lassa virus, Machupo virus, Sabiá virus, Crimean-Congo hemorrhagic fever virus, Ebola virus, Marburg virus, Measles virus, Mumps virus, Parainfluenza virus, Respiratory syncytial virus, Human metapneumovirus, Hendra virus, Nipah virus, Rabies virus, Hepatitis D, Rotavirus, Orbivirus, Coltivirus, Banna virus, and Merkel cell polyomavirus.

20. The composition of claim 13, wherein the vector comprises one selected from the group consisting of a plasmid, a nanoparticle, a cationic lipid, a cationic polymer, a metallic nanoparticle, a nanorod, a liposome, a micelle, a microbubble, a cell-penetrating peptide, and a liposphere.

21. The composition of claim 13, wherein the vector is a viral vector.

22. The composition of claim 13, wherein the vector also encodes the treatment.

23. A method for targeted cutting of viral nucleic acid, the method comprising:
introducing into a host cell: a nuclease, a targeting sequence that targets the nuclease to the viral nucleic acid, and a DNA repair inhibitor;
targeted cutting, by the nuclease, of the viral nucleic acid into fragments; and
preventing, via the DNA repair inhibitor, ligation of ends of the fragments.

24. The method of claim 23, wherein the nuclease and the targeting sequence are introduced using a vector that encodes the nuclease and the targeting sequence.

25. The method of claim 24, wherein the vector also encodes the DNA repair inhibitor.

26. The method of claim 23, wherein the DNA repair inhibitor inhibits homologous and non-homologous end repair of the fragments.

27. The method of claim 26, wherein the DNA repair inhibitor is selected from a chain-terminating nucleotide, chain-terminating nucleotide analogue, a chain-terminating nucleoside, a chain-terminating nucleoside analogue, and a phosphatase.

28. The method of claim 23, wherein the nuclease is selected from the group consisting of a zinc-finger nuclease, a transcription activator-like effector nuclease, a meganuclease, and a Cas9 endonuclease.
